(19) United States
(12) Patent Application Publication
(10) Pub. No.: US 2026/0137847 A1
(43) Pub. Date: May 21, 2026

(54) Breast Pump, and Self-Suction Breast Pumping Device, Self-Suction Cover Thereof and Operation Method Therefor

(71) Applicant: NINGBO XIAOWU BRAND MANAGEMENT CO., LTD., Ningbo (CN)

(72) Inventors: Qiansheng Ye, Ningbo (CN); Yongliang Wang, Ningbo (CN); Jinwei Xin, Ningbo (CN)

(21) Appl. No.: 19/450,661

(22) Filed: Jan. 15, 2026

Related U.S. Application Data
(63) Continuation of application No. PCT/CN2024/120260, filed on Sep. 23, 2024.

(30) Foreign Application Priority Data
Nov. 14, 2023 (CN) 202323080630.8
Jul. 10, 2024 (CN) 202410923768.X
Jul. 10, 2024 (CN) 202421628726.5
Sep. 23, 2024 (WO) PCT/CN2024/120260

(51) Int. Cl. A61M 1/06; A61M 39/00; A61M 39/24

(52) U.S. Cl. CPC A61M 1/066; A61M 1/067; A61M 1/069; A61M 39/24; A61M 2039/0009; A61M 2202/0014; A61M 2202/04; A61M 2205/3331; A61M 2210/1007

(57) ABSTRACT

A self-suction breast pumping device includes a milk discharge one-way valve, a self-suction cover including an assembly ring and a horn cover, and a negative pressure forming unit, wherein the negative pressure forming unit has a negative pressure chamber, an air exchange channel communicated with the negative pressure chamber, a milk suction chamber independent of the negative pressure chamber, and an assembly opening and a milk discharge channel communicated with the milk suction chamber, wherein a volume size of the negative pressure chamber is negatively correlated with a volume size of the milk suction chamber, wherein the milk discharge one-way valve is disposed in the negative pressure forming unit for selectively opening and closing the milk discharge channel of the negative pressure forming unit, wherein the assembly ring of the self-suction cover is assembled to the negative pressure forming unit at the assembly opening.

### CROSS REFERENCE OF RELATED APPLICATION

This is a Continuation Application of PCT/CN 2024/120260, filed Sep. 23, 2024, which claims priority under 35 U.S.C. 119(a-d) to Chinese application numbers CN 202323080630.8, filed Nov. 14, 2023; CN 202410923768.X, filed Jul. 10, 2024 and CN 202421628726.5, filed Jul. 10, 2024, the afore-mentioned patent applications are hereby incorporated by reference in their entireties.

### BACKGROUND OF THE PRESENT INVENTION

### Field of Invention

The present invention relates to the technical field of maternal and infant products, and more particularly to a breast pump, a self-suction breast pumping device, its self-suction cover, and its operating method.

### DESCRIPTION OF RELATED ARTS

In recent years, wearable breast pumps have become increasingly popular to assist breastfeeding mothers with milk expression. Conventional wearable breast pumps allow users to place them inside a bra, which holds them in place on the breast, allowing for pumping while walking without hand intervention. However, conventional wearable breast pumps still have several drawbacks. For example, they require bra support for fixation, which not only increases the inconvenience of use but can also affect pumping efficiency if the bra is ill-fitting or worn improperly. Furthermore, the bra-assisted method of securing the pump to the breast can cause additional pressure, leading to discomfort during pumping and potentially impacting breast health with prolonged use. Therefore, the inventors of this invention have dedicated themselves to developing a breast pump that can be fixed in place on the breast without any assistance.

### SUMMARY OF THE PRESENT INVENTION

An object of the present invention is to provide a breast pump, a self-suction breast pumping device, a self-suction cover and an operating method thereof, wherein the self-suction breast pumping device can be attached to the breast without the need for underwear, so as to achieve breast pumping without underwear.

An object of the present invention is to provide a breast pump, a self-suction breast pumping device, a self-suction cover and an operating method thereof, wherein the self-suction breast pumping device can form a negative pressure space between the horn cover of the self-suction cover and the breast, thereby enabling the self-suction breast pumping device to be attached to the breast without the need for underwear.

An object of the present invention is to provide a breast pump, a self-suction breast pumping device, a self-suction cover, and an operating method thereof, wherein during the process of a user attaching the self-suction breast pumping device to the breast, the horn cover is first flipped over, then the position of the horn cover for connecting the assembly ring is allowed to contact the areola, and then the horn cover is restored so that the horn cover covers a portion of the breast. In this way, the self-suction breast pumping device can form the negative pressure space between the horn cover and the breast, so that the self-suction breast pumping device can be attached to the breast without the need for a bra.

An object of the present invention is to provide a breast pump, a self-suction breast pumping device, a self-suction cover and an operating method thereof, wherein the air in the negative pressure space can be discharged through an exhaust one-way valve, thereby enabling the self-suction breast pumping device to reliably adhere to the breast.

According to one aspect of the present invention, the present invention provides a method for operating a self-adsorption type breast pump, wherein the method comprises the following steps:
(a) flipping a horn cover of a self-suction cover toward a negative pressure forming unit;
(b) allowing a position of the horn cover of the self-suction cover for connecting an assembly ring to abut an areola; and
(c) restoring the horn cover so that the horn cover covers a portion of a breast to create a negative pressure space between the horn cover and the breast, so as to allow the self-suction breast pumping device to attach to the breast.

According to an embodiment of the present invention, in the step (b), an inner wall of a small cover of the self-suction cover is allowed to conform to the areola in such a way that the small cover covers a nipple, wherein the small cover is extended from the position of the horn cover for connecting the assembly ring.

According to an embodiment of the present invention, in the step (b), a contact area between the horn cover of the self-suction cover and an upper part of the breast is greater than a contact area between the horn cover and a lower part of the breast.

According to an embodiment of the present invention, in the above method, the negative pressure space formed between the horn cover and the breast is allowed to be communicated to a milk suction chamber of the negative pressure forming unit through an exhaust one-way valve, wherein the exhaust one-way valve allows air in the negative pressure space to flow toward the milk suction chamber and prevents air in the milk suction chamber from flowing toward the negative pressure space.

According to an embodiment of the present invention, in the above method, the negative pressure space formed between the horn cover of the self-suction cover and the breast is allowed to be communicated to an external environment of the negative pressure forming unit through an exhaust one-way valve, wherein the exhaust one-way valve allows air in the negative pressure space to flow to the outside and prevents air from the external environment from flowing toward the negative pressure space.

According to an embodiment of the present invention, in the above method, the exhaust one-way valve is extended into a valve chamber located at a top of the negative pressure forming unit, a connecting channel of the negative pressure forming unit connects the valve chamber with the milk suction chamber, wherein an air flow rate at a connecting position between the connecting channel and the valve chamber is less than an air flow rate at a connecting position between the connecting channel and the milk suction chamber.

According to another aspect of the present invention, the present invention provides a self-suction breast pumping device, comprising;
a milk discharge one-way valve;
a self-suction cover, wherein the self-suction cover comprises an assembly ring and a horn cover which is deformable and extended outward from the assembly ring; and
a negative pressure forming unit, wherein the negative pressure forming unit has a negative pressure chamber, an air exchange channel communicated with the negative pressure chamber, a milk suction chamber independent of the negative pressure chamber, and an assembly opening and a milk discharge channel communicated with the milk suction chamber, wherein a volume size of the negative pressure chamber is negatively correlated with a volume size of the milk suction chamber, wherein the milk discharge one-way valve is disposed in the negative pressure forming unit for selectively opening and closing the milk discharge channel of the negative pressure forming unit, wherein the assembly ring of the self-suction cover is assembled to the negative pressure forming unit at the assembly opening.

According to an embodiment of the present invention, the horn cover comprises a rear portion and a front portion, the rear portion of the horn cover connects the front portion of the horn cover with the assembly ring, wherein an angle α is defined between an extending direction of the rear portion of the horn cover and a central axis of the self-suction cover, and an angle β is defined between an extending direction of the front portion of the horn cover and the central axis of the self-suction cover, wherein the angles α and β satisfy: α>β.

According to an embodiment of the present invention, the self-suction cover further comprises a small cover which is disposed at a connection position between the horn cover and the assembly ring, and at least a portion of the small cover body is surrounded by the horn cover.

According to an embodiment of the present invention, the horn cover has an exhaust passage, wherein the self-suction cover comprises an exhaust one-way valve disposed on the horn cover for selectively opening and closing the exhaust passage of the horn cover.

According to an embodiment of the present invention, an end of the exhaust one-way valve opposite to the horn cover is located outside the negative pressure forming unit.

According to an embodiment of the present invention, the negative pressure forming unit has a valve chamber and a connecting channel, the valve chamber is located above the milk suction chamber, the connecting channel connects the valve chamber with the milk suction chamber, wherein the end of the exhaust one-way valve away from the horn cover is extended into the valve chamber of the negative pressure forming unit.

According to an embodiment of the present invention, an air flow rate at a connection position between the connecting channel and the valve chamber is less than an air flow rate at a connection position between the connecting channel and the milk suction chamber.

According to an embodiment of the present invention, a distance between a front side of a top of the horn cover and a rear side of the assembly ring is greater than a distance between a front side of a bottom of the horn cover and the rear side of the assembly ring.

According to another aspect of the present invention, the present invention provides a breast pump comprising a main unit, a self-suction breast pumping device, and an air tube connecting the main unit with the self-suction breast pumping device, wherein the self-suction breast pumping device comprises:
a milk discharge one-way valve;
a self-suction cover, wherein the self-suction cover comprises an assembly ring and a horn cover which is deformable and extended outward from the assembly ring; and
a negative pressure forming unit, wherein the negative pressure forming unit has a negative pressure chamber, an air exchange channel communicated with the negative pressure chamber, a milk suction chamber independent of the negative pressure chamber, and an assembly opening and a milk discharge channel communicated with the milk suction chamber, wherein a volume size of the negative pressure chamber is negatively correlated with a volume size of the milk suction chamber, wherein the milk discharge one-way valve is disposed in the negative pressure forming unit for selectively opening and closing the milk discharge channel of the negative pressure forming unit, wherein the assembly ring of the self-suction cover is assembled to the negative pressure forming unit at the assembly opening.

According to another aspect of the present invention, the present invention further provides a self-suction cover, comprising:
an assembly ring; and
a horn cover which is deformable, wherein the horn cover comprises a rear portion and a front portion, the rear portion of the horn cover connects the front portion of the horn cover with the assembly ring, wherein an angle α is defined between an extending direction of the rear portion of the horn cover and a central axis of the self-suction cover, and an angle β is defined between an extending direction of the front portion of the horn cover and the central axis of the self-suction cover, wherein the angles α and β satisfy: α>β.

According to an embodiment of the present invention, the included angle β satisfies:
15°≤β≤40°.

According to an embodiment of the present invention, the distance between the front side of the top of the horn cover and the rear side of the assembly ring is greater than the distance between the front side of the bottom of the horn cover and the rear side of the assembly ring.

According to an embodiment of the present invention, the horn cover has an exhaust passage, wherein the self-suction cover comprises an exhaust one-way valve, the exhaust one-way valve is disposed on the horn cover for selectively opening and closing the exhaust passage of the horn cover.

According to an embodiment of the present invention, the exhaust one-way valve and the horn cover are integrally formed.

According to an embodiment of the present invention, the self-suction cover further comprises a small cover disposed at the connection position of the horn cover body and the assembly ring body, and at least a portion of the small cover body is surrounded by the horn cover body.

According to another aspect of the present invention, the present invention further provides a self-suction cover, comprising:
an assembly ring;
a deformable horn cover, wherein the horn cover is extended integrally outward from the assembly ring and has an exhaust passage; and
an exhaust one-way valve, wherein the exhaust one-way valve is disposed on the horn cover for selectively opening and closing the exhaust passage of the horn cover.

According to another aspect of the present invention, the present invention further provides a self-suction breast pumping device, comprising:
a milk discharge one-way valve;
a self-suction cover, wherein the self-suction cover comprises an assembly ring and a horn cover which is deformable and extended outward from the assembly ring; and
a negative pressure forming unit, wherein the negative pressure forming unit has a negative pressure chamber, an air exchange channel communicated with the negative pressure chamber, a milk suction chamber independent of the negative pressure chamber, and an assembly opening and a milk discharge channel communicated with the milk suction chamber, wherein a volume size of the negative pressure chamber is negatively correlated with a volume size of the milk suction chamber, wherein the milk discharge one-way valve is disposed in the negative pressure forming unit for selectively opening and closing the milk discharge channel of the negative pressure forming unit, wherein the assembly ring of the self-suction cover is assembled to the negative pressure forming unit at the assembly opening. According to an embodiment of the present invention, wherein the horn cover comprises a rear portion and a front portion, the rear portion of the horn cover connects the front portion of the horn cover with the assembly ring, wherein an angle α is defined between an extending direction of the rear portion of the horn cover and a central axis of the self-suction cover, and an angle β is defined between an extending direction of the front portion of the horn cover and the central axis of the self-suction cover, wherein the angles α and β satisfy: α>β.

According to an embodiment of the present invention, the self-suction cover further comprises a small cover disposed at the connection position of the horn cover and the assembly ring, and at least a portion of the small cover body is surrounded by the horn cover body.

According to an embodiment of the present invention, the rear side of the assembly ring is extended above the opening of the milk discharge channel of the negative pressure forming unit.

According to an embodiment of the present invention, the horn cover has an exhaust passage, wherein the self-suction cover comprises an exhaust one-way valve, the exhaust one-way valve is disposed on the horn cover for selectively opening and closing the exhaust passage of the horn cover.

According to an embodiment of the present invention, the end of the exhaust one-way valve opposite to the horn cover is located outside the negative pressure forming unit.

According to an embodiment of the present invention, the negative pressure forming unit has a valve chamber and a connecting channel, the valve chamber is located above the milk suction chamber, the connecting channel connecting the valve chamber and the milk suction chamber, wherein one end of the exhaust one-way valve facing away from the horn cover is extended into the valve chamber of the negative pressure forming unit.

According to an embodiment of the present invention, the air flow rate at the connection position between the connecting channel and the valve chamber is less than the air flow rate at the connection position between the connecting channel and the milk suction chamber.

According to an embodiment of the present invention, the distance between the front side of the top of the horn cover and the rear side of the assembly ring is greater than the distance between the front side of the bottom of the horn cover and the rear side of the assembly ring.

According to an embodiment of the present invention, the negative pressure forming unit comprises a supporting housing and a negative pressure forming part. The supporting housing has a housing cavity and a housing opening and a housing notch respectively communicated with the housing cavity. The negative pressure forming part comprises a deformable bowl, an annular frame disposed at the opening of the deformable bowl, and a mounting arm extended downward from the frame. The frame is mounted to the supporting housing at the housing opening. The deformable bowl is extended into the housing cavity of the supporting housing. The mounting arm is located at the housing notch of the supporting housing. The negative pressure chamber of the negative pressure forming unit is formed between the supporting housing and the deformable bowl. The air exchange channel of the negative pressure forming unit is formed in the supporting housing. The milk suction chamber and the assembly opening of the negative pressure forming unit are formed in the frame. The milk discharge channel of the negative pressure forming unit is extended from the inner wall of the deformable bowl through the frame to the end of the mounting arm or from the inner wall of the frame to the end of the mounting arm. The milk discharge one-way valve is mounted on the mounting arm.

According to an embodiment of the present invention, the self-suction cover further comprises a limiting portion extended from the peripheral wall of the assembly ring in a direction substantially perpendicular to the extending direction of the assembly ring, and a gap is formed between the limiting portion and the horn cover, wherein the limiting portion is capable of abutting against the frame of the negative pressure forming part.

According to an embodiment of the present invention, the supporting housing comprises a housing body and an outer cover, and the supporting housing has a housing receiving cavity. The housing cavity, the housing opening, and the housing notch of the supporting housing are formed in the housing body. The outer cover is installed on the housing body. The supporting housing forms the housing receiving cavity between the housing body and the outer cover. The outer cover has a positioning hole that is communicated with the housing receiving cavity of the supporting housing. The negative pressure forming unit comprises an airway unit. The airway unit comprises an air guide base and a reversing base. The air guide base has a laterally extending first air passage, a bottom insertion hole, a top insertion hole, and a top boss. The bottom insertion hole and the top insertion hole are respectively communicated with the first air passage, and the opening of the top insertion hole is formed on the end face of the top boss. The housing body comprises a housing insertion arm extended into the housing receiving cavity of the supporting housing. The opening of the air exchange channel of the negative pressure forming unit is formed on the end face of the housing insert arm of the housing body. The air guide base is disposed on the top of the housing body. The housing insert arm of the housing body is inserted into the bottom insertion hole of the air guide base. The top boss of the air guide base is extended into the positioning hole of the outer cover. The air guide base is received in the housing receiving cavity of the supporting housing. The first air passage of the air guide base is communicated to the air exchange channel of the negative pressure forming unit. The reversing base comprises a bottom insert arm, a side insert arm, and a second air passage. One opening of the second air passage is formed on the end face of the bottom insert arm, and the other opening is formed on the end face of the side insert arm. The bottom insert arm of the reversing base is inserted into the top insertion hole of the air guide base so that the negative pressure chamber of the negative pressure forming unit and the air exchange channel, the first air passage of the air guide base, and the second air passage of the reversing base are sequentially connected.

According to another aspect of the present invention, the present invention further provides a breast pump, which comprises a main unit, an air tube, and a self-suction breast pumping device, wherein the air tube connects the main unit with the self-suction breast pumping device, and wherein the self-suction breast pumping device further comprises:
a milk discharge one-way valve;
a self-suction cover, wherein the self-suction cover comprises an assembly ring and a horn cover which is deformable and extended outward from the assembly ring; and
a negative pressure forming unit, wherein the negative pressure forming unit has a negative pressure chamber, an air exchange channel communicated with the negative pressure chamber, a milk suction chamber independent of the negative pressure chamber, and an assembly opening and a milk discharge channel communicated with the milk suction chamber, wherein a volume size of the negative pressure chamber is negatively correlated with a volume size of the milk suction chamber, wherein the milk discharge one-way valve is disposed in the negative pressure forming unit for selectively opening and closing the milk discharge channel of the negative pressure forming unit, wherein the assembly ring of the self-suction cover is assembled to the negative pressure forming unit at the assembly opening.

### BRIEF DESCRIPTION OF THE DRAWINGS

FIG. 1 is a schematic view of a breast pump according to a preferred embodiment of the present invention.

FIG. 2 is a perspective view of a self-suction breast pumping device of the breast pump according to the above mentioned preferred embodiment of the present invention.

FIG. 3 is another perspective view of the self-suction breast pumping device of the breast pump according to the above mentioned preferred embodiment of the present invention.

FIG. 4 is a cross-sectional view of the self-suction breast pumping device of the breast pump according to the above mentioned preferred embodiment of the present invention.

FIG. 5 is a schematic view illustrating a first step of the processes in which the self-suction breast pumping device of the breast pump according to the above mentioned preferred embodiment of the present invention is attached to the breast.

FIG. 6 is a schematic view illustrating a second step of the process in which the self-suction breast pumping device of the breast pump according to the above mentioned preferred embodiment of the present invention is attached to the breast.

FIG. 7 is a schematic view illustrating a third step of the process in which the self-suction breast pumping device of the breast pump according to the above mentioned preferred embodiment of the present invention is attached to the breast, wherein the state in which the self-suction breast pumping device is attached to the breast is shown.

FIGS. 8A and 8B are perspective views of different views of a partial structure of a modified example of the self-suction breast pumping device of the breast pump according to the above mentioned preferred embodiment of the present invention.

FIGS. 9A and 9B are exploded schematic views from different perspectives of the partial structure of the self-suction breast pumping device of the breast pump according to the above mentioned preferred embodiment of the present invention.

FIG. 10 is a schematic cross-sectional view of a partial structure of a modified example of the self-suction breast pumping device of the breast pump according to the above mentioned preferred embodiment of the present invention.

FIG. 11 is a partial structural schematic view of a modified example of the self-suction breast pumping device of the breast pump according to the above mentioned preferred embodiment of the present invention.

FIG. 12 is a schematic cross-sectional view of a partial structure of a modified example of the self-suction breast pumping device of the breast pump according to the above mentioned preferred embodiment of the present invention.

### DETAILED DESCRIPTION OF THE PREFERRED EMBODIMENT

Before detailing any embodiment of the invention, it should be understood that the invention, in its application, is not limited to the details of the construction and arrangement of the components set forth in the following description or illustrated in the following FIGS. The invention can have other embodiments and can be practiced or carried out in various ways. Furthermore, it should be understood that the wording and terminology used herein are for descriptive purposes and should not be considered limiting. The use of “comprising” or “having” and variations thereof is intended to cover the items set forth below and their equivalents, as well as any additional items. Unless otherwise specified or limited, the terms “installation”, “connection”, “support”, and “linkage”, and variations thereof are used broadly and cover both direct and indirect installation, connection, support, and linking. Moreover, “connect” and “link” are not limited to physical or mechanical connections or links.

Furthermore, firstly, in the disclosure of this invention, the terms “longitudinal”, “lateral”, “upper”, “lower”, “front”, “rear”, “left”, “right”, “vertical”, “horizontal”, “top”, “bottom”, “inner”, and “outer”, etc., indicate the orientation or positional relationship based on the orientation or positional relationship shown in the accompanying drawings. They are only for the convenience of describing this invention and simplifying the description, and do not indicate or imply that the device or element referred to must have a specific orientation, or be constructed and operated in a specific orientation. Therefore, the above terms should not be construed as limiting the invention. Secondly, the term “a” or “an” should be understood as “at least one” or “one or more”, that is, in one embodiment, the number of an element can be one, while in another embodiment, the number of the element can be multiple. The term “a” or “an” should not be construed as a limitation on the quantity.

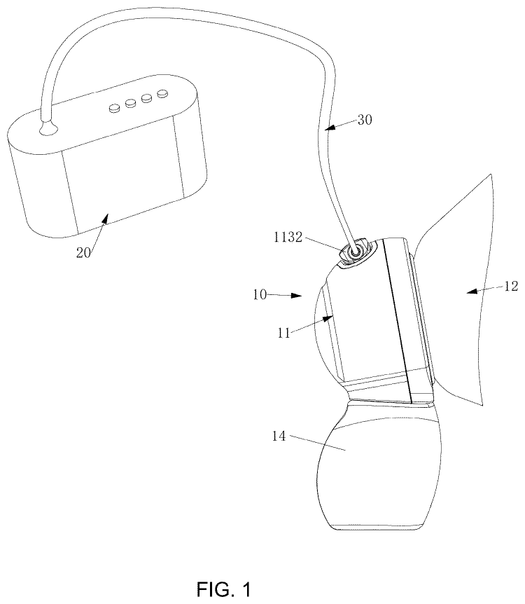

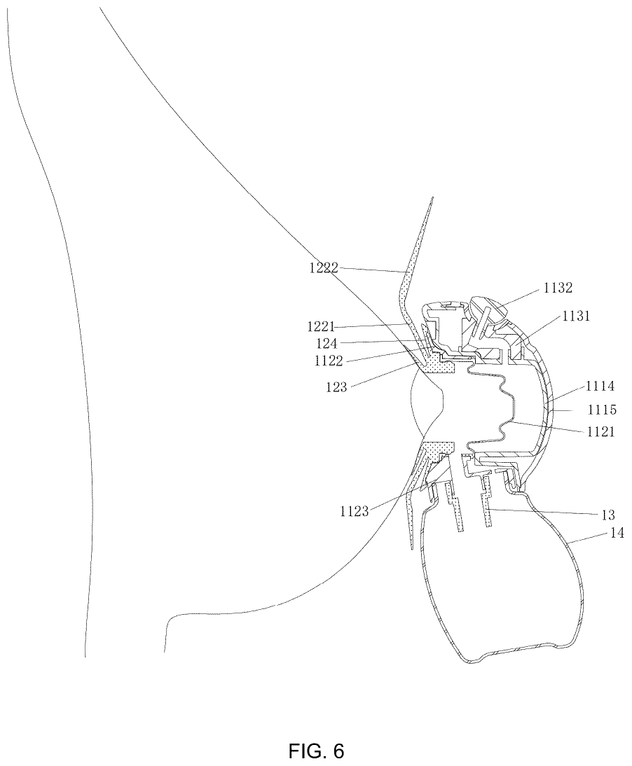

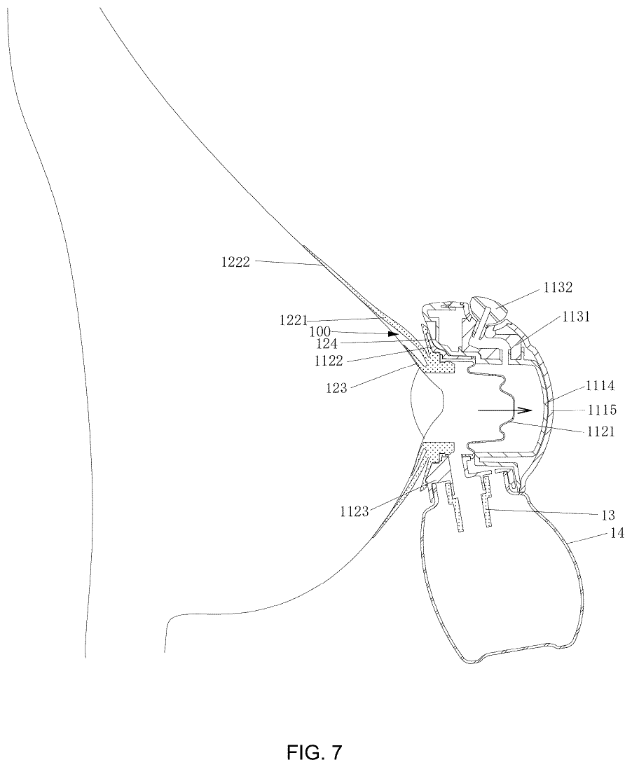

Referring to FIGS. 1 to 7 of the accompanying drawings of this invention, a breast pump according to an embodiment of the present invention is disclosed and described below. The breast pump comprises at least one self-suction breast pumping device 10, a main unit 20, and an air tube 30 connecting the self-suction breast pumping device 10 and the main unit 20. After the self-suction breast pumping device 10 is adsorbed onto the breast, the main unit 20 assists the user in pumping milk. Preferably, there are two self-suction breast pumping devices 10, so that the two self-suction breast pumping devices 10 can be adsorbed onto two breasts for simultaneous pumping.

In this specific example of the breast pump of the present invention shown in FIGS. 1 to 7, the breast pump allows the self-suction breast pumping device 10 to be attached to the breast without the need for underwear, so as to enable “naked pumping”. It should be noted that “naked pumping” as described in the breast pump of the present invention means that the self-suction breast pumping device 10 is directly attached to the breast without the need for underwear or any other aids, and milk is pumped while the main unit 20 is turned on. Nevertheless, the breast pump of the present invention also allows the user to use it while wearing underwear. In this case, a portion of the self-suction breast pumping device 10 is positioned between the underwear and the breast, which significantly reduces the size of the portion of the self-suction breast pumping device 10 located between the underwear and the breast, thereby reducing pressure on the breast. This not only facilitates milk pumping but also promotes breast health.

Specifically, referring to FIGS. 1 to 7, the self-suction breast pumping device 10 comprises a negative pressure forming unit 11, a self-suction cover 12, and a milk discharge one-way valve 13. The self-suction cover 12 and the milk discharge one-way valve 13 are respectively disposed at the front and bottom of the negative pressure forming unit 11, and the negative pressure forming unit 11 is connected to the main unit 20. When the user attaches the self-suction breast pumping device 10 to the breast through the self-suction cover 12, a negative pressure space 100 is formed between the self-suction cover 12 and the breast, thereby enabling the self-suction breast pumping device 10 to be attached to the breast without the need for underwear.

More specifically, the negative pressure forming unit 11 has a negative pressure chamber 1101, an air exchange channel 1102, a milk suction chamber 1103, an assembly opening 1104, and a milk discharge channel 1105. The air exchange channel 1102 is communicated to the negative pressure chamber 1101. The main unit 20 can change the air pressure state of the negative pressure chamber 1101 through the air exchange channel 1102. That is, the main unit 20 can increase and decrease the air pressure of the negative pressure chamber 1101 through the air exchange channel 1102 of the negative pressure forming unit 11. The assembly opening 1104 and the milk discharge channel 1105 are respectively communicated to the milk suction chamber 1103. The negative pressure chamber 1101 and the milk suction chamber 1103 of the negative pressure forming unit 11 are independent of each other, and the size of the negative pressure chamber 1101 and the size of the milk suction chamber 1103 are negatively correlated. When the space of the negative pressure chamber 1101 of the negative pressure forming unit 11 increases, the space of the milk suction chamber 1103 decreases. Correspondingly, when the space of the negative pressure chamber 1101 of the negative pressure forming unit 11 decreases, the space of the milk suction chamber 1103 increases. Furthermore, the air pressure state of the negative pressure chamber 1101 of the negative pressure forming unit 11 is positively correlated with the air pressure state of the milk suction chamber 1103. When the air pressure of the negative pressure chamber 1101 of the negative pressure forming unit 11 increases, the air pressure of the milk suction chamber 1103 increases, and when the air pressure of the negative pressure chamber 1101 of the negative pressure forming unit 11 decreases, the air pressure of the milk suction chamber 1103 decreases.

The self-suction cover 12 comprises an assembly ring 121 and a deformable horn cover 122 extended outward from the assembly ring 121. The assembly ring 121 of the self-suction cover 12 is assembled to the negative pressure forming unit 11 at the assembly opening 1104. The milk discharge one-way valve 13 is disposed in the negative pressure forming unit 11 at the milk discharge channel 1105 of the negative pressure forming unit 11 to selectively open and close the milk discharge channel 1105 of the negative pressure forming unit 11.

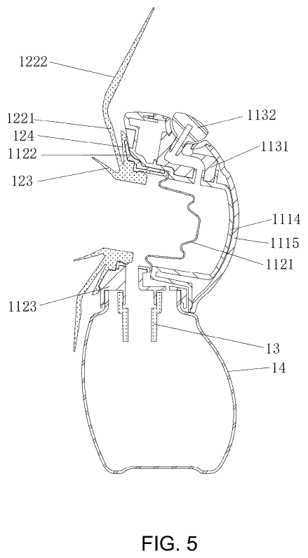

Referring to FIGS. 5 to 7, the process of attaching the self-suction breast pumping device 10 to the breast can be as follows: first, the horn cover 122 of the self-suction cover 12 is flipped toward the negative pressure forming unit 11; second, the position of the horn cover 122 for connecting the assembly ring 121 is allowed to contact the areola; third, the horn cover 122 is restored so that it covers part of the breast, thereby forming a negative pressure space 100 between the horn cover 122 and the breast, so as to allow the self-suction breast pumping device 10 to attach to the breast and achieve naked breast pumping. In other words, after the self-suction breast pumping device 10 is adsorbed onto the breast, the breast closes the front opening of the horn cover 122, and the areola closes the rear opening of the horn cover 122. In this way, the self-suction breast pumping device 10 not only allows the negative pressure space 100 to be formed between the horn cover 122 and the breast, but also allows the milk suction chamber 1103 of the negative pressure forming unit 11 to form a closed space, which helps to ensure milk suction efficiency.

During the milk pumping process, when the main unit 20 inhales, it allows the air in the negative pressure chamber 1101 of the negative pressure forming unit 11 to be discharged through the air exchange channel 1102, thereby reducing the air pressure and space of the negative pressure chamber 1101. At this time, the space of the milk suction chamber 1103 of the negative pressure forming unit 11 increases and the air pressure decreases, so that milk is sucked out of the breast and temporarily stored in the milk suction chamber 1103 of the negative pressure forming unit 11. Simultaneously, during this process, the areola tends to be pulled toward the milk suction chamber 1103 of the negative pressure forming unit 11. The position of the horn cover 122 for connecting the assembly ring 121 prevents the areola from being pulled toward the milk suction chamber 1103 of the negative pressure forming unit 11. This allows the position of the horn cover 122 for connecting the assembly ring 121 to squeeze the areola to squeeze out the milk. When the main unit 20 vents air, it allows air to be added to the negative pressure chamber 1101 of the negative pressure forming unit 11, thereby increasing the air pressure and space of the negative pressure chamber 1101. At this time, the space of the milk suction chamber 1103 of the negative pressure forming unit 11 decreases and the air pressure increases. The milk discharge one-way valve 13 automatically opens to discharge the milk temporarily stored in the milk suction chamber 1103 of the negative pressure forming unit 11. This process is repeated, and the breast pump achieves milk suction.

In other words, the space of the milk suction chamber 1103 of the negative pressure forming unit 11 decreases as the space of the negative pressure chamber 1101 increases and increases as the space of the negative pressure chamber 1101 decreases, thereby achieving that the air pressure in the milk suction chamber 1103 of the negative pressure forming unit 11 increases when the air pressure in the negative pressure chamber 1101 increases and decreases when the air pressure in the negative pressure chamber 1101 decreases.

Furthermore, the self-suction breast pumping device 10 comprises a milk bottle 14 which is installed to the negative pressure forming unit 11. The opening of the milk discharge one-way valve 13 faces toward the milk bottle 14. In this way, after the milk discharge one-way valve 13 is automatically opened, the milk temporarily stored in the milk suction chamber 1103 of the negative pressure forming unit 11 can be collected into the milk bottle 14.

Referring again to FIGS. 1 to 7, the negative pressure forming unit 11 comprises a supporting housing 111 and a negative pressure forming part 112. The supporting housing 111 has a housing cavity 1111, and a housing opening 1112 and a housing notch 1113 respectively communicated with the housing cavity 1111. The negative pressure forming part 112 comprises a deformable bowl 1121, an annular frame 1122 disposed at the opening of the deformable bowl 1121, and a mounting arm 1123 extended downward from the frame 1122. The frame 1122 is mounted on the supporting housing 111 at the housing opening 1112. The deformable bowl 1121 is extended into the housing cavity 1111 of the supporting housing 111. The mounting arm 1123 is located at the housing notch 1113 of the supporting housing 111. The negative pressure chamber 1101 of the negative pressure forming unit 11 is formed between the supporting housing 111 and the deformable bowl 1121. The air exchange channel 1102 of the negative pressure forming unit 11 is formed in the supporting housing 111. The milk suction chamber 1103 and the assembly opening 1104 of the negative pressure forming unit 11 are formed in the frame 1122. The milk discharge channel 1105 of the negative pressure forming unit 11 is extended from the inner wall of the deformable bowl 1121 through the frame 1122 to the end of the mounting arm 1123. Alternatively, in other examples of the breast pump of the present invention, the milk discharge channel 1105 of the negative pressure forming unit 11 may be extended from the inner wall of the frame 1122 to the end of the mounting arm 1123.

In other words, whether the opening of the milk discharge channel 1105 of the negative pressure forming unit 11 is formed on the inner wall of the deformable bowl 1121 or on the inner wall of the frame 1122 depends on the width of the joint between the deformable bowl 1121 and the frame 1122. In embodiments where the width of the joint between the deformable bowl 1121 and the frame 1122 is wider, the opening of the negative pressure forming unit 1105 is formed on the inner wall of the deformable bowl 1121. In embodiments where the width of the joint between the deformable bowl 1121 and the frame 1122 is narrower, the opening of the negative pressure forming unit 1105 is formed on the inner wall of the frame 1122.

It is also worth mentioning that the specific manner in which the frame 1122 is disposed at the opening of the deformable bowl 1121 is not limited to the breast pump of the present invention. For example, in some embodiments, after the frame 1122 and the deformable bowl 1121 are respectively molded, the frame 1122 is glued to the opening of the deformable bowl 1121; in other embodiments, based on a secondary injection molding process, the frame 1122 may also be disposed at the opening of the deformable bowl 1121.

Both the milk discharge one-way valve 13 and the milk bottle 14 are mounted on the mounting arm 1123 of the negative pressure forming part 112. Specifically, the mounting arm 1123 of the negative pressure forming part 112 comprises an inner arm body 11231 and an outer arm body 11232 surrounding the inner arm body 11231. The milk discharge one-way valve 13 is mounted on the inner arm body 11231, and the milk bottle 14 is mounted on the outer arm body 11232. Thus, both the milk discharge one-way valve 13 and the milk bottle 14 are mounted on the mounting arm 1123, and the opening of the milk discharge one-way valve 13 faces toward the milk bottle 14.

When the air pressure in the negative pressure chamber 1101 of the negative pressure forming unit 11 changes, the shape of the deformable bowl 1121 of the negative pressure forming part 112 changes accordingly, and simultaneously the air pressure and space size of the milk suction chamber 1103 of the negative pressure forming unit 11 change. Specifically, when the air pressure in the negative pressure chamber 1101 of the negative pressure forming unit 11 decreases, the deformable bowl 1121 of the negative pressure forming part 112 deforms toward the bottom of the housing cavity 1111 of the supporting housing 111 to reduce the air pressure in the milk suction chamber 1103 of the negative pressure forming unit 11. Correspondingly, when the air pressure in the negative pressure chamber 1101 of the negative pressure forming unit 11 increases, the deformable bowl 1121 of the negative pressure forming part 112 deforms toward the bottom of the housing cavity 1111 of the supporting housing 111 to increase the air pressure in the milk suction chamber 1103 of the negative pressure forming unit 11.

It is worth mentioning that when the air pressure in the negative pressure chamber 1101 of the negative pressure forming unit 11 increases, the reason why the deformable bowl 1121 of the negative pressure forming part 112 can deform toward the bottom of the housing cavity 1111 away from the supporting housing 111 is because the deformable bowl 1121 of the negative pressure forming part 112 accumulates elastic potential energy after deforming toward the bottom of the housing cavity 1111 closer to the supporting housing 111.

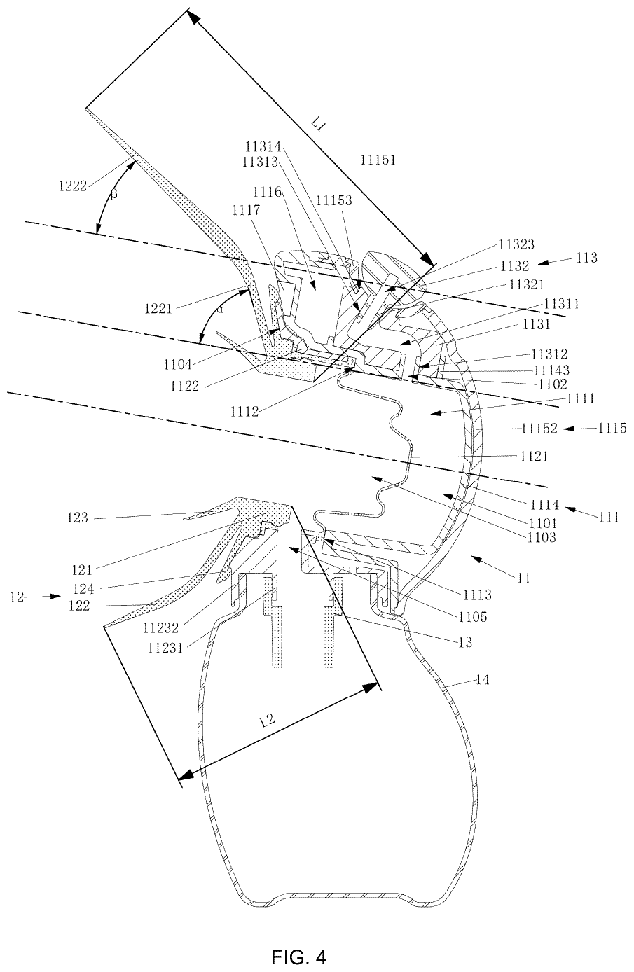

Referring to FIG. 4, the horn cover 122 of the self-suction cover 12 comprises a rear portion 1221 and a front portion 1222. The rear portion 1221 connects the front portion 1222 and the assembly ring 121. The extension direction of the rear portion 1221 forms an angle α with the central axis of the self-suction cover 12, and the extension direction of the front portion 1222 forms an angle β with the central axis of the self-suction cover 12. Angles α and β satisfy: α>β. In this way, on the one hand, the front side of the horn cover 122 can have a larger opening, and on the other hand, the rear side of the horn cover 122 (i.e., the position of the horn cover 122 for connecting the assembly ring 121) can also be larger. The device can have a small opening, so that when the self-suction breast pumping device 10 is adsorbed onto the breast, a negative pressure space 100 can be formed between the horn cover 122 and the breast. On the other hand, the center of gravity of the self-suction breast pumping device 10 can be closer to the self-suction cover 12, so that after the self-suction breast pumping device 10 is adsorbed onto the breast, the center of gravity of the self-suction breast pumping device 10 is closer to the body, thereby reducing the component of gravity of the self-suction breast pumping device 10 acting on the breast. This not only reduces the adsorption force required by the self-suction breast pumping device 10 on the self-suction cover 12, but also avoids the self-suction breast pumping device 10 from compressing the breast during the breast pumping process, thus ensuring breast health.

It is understandable that the angle β between the extending direction of the front portion 1222 of the horn cover 122 and the central axis of the self-suction cover 12 cannot be too small or too large. If the angle β is too small, the opening size of the horn cover 122 will be too small, which will result in the horn cover 122 not being able to cover the breast and the position of the horn cover 122 used to connect the assembly ring 121 not touching the areola. Even if the breast is forcibly inserted into the horn cover 122, the horn cover 122 will provide a large clamping force and act on the breast, causing breast discomfort or even damage. If the angle β is too large, the opening size of the horn cover 122 will be too large, which will result in the horn cover 122 not fitting the breast and the self-suction cover 12 not being able to attach to the breast. In the self-suction breast pumping device 10 of the present invention, the minimum value of the included angle β is preferably not less than 15, and the maximum value is preferably not greater than 40. Therefore, the suitable range of the included angle β is 15° to 40°. In other words, the included angle β satisfies: 15°≤β≤40°.

Furthermore, the hardness range of the horn cover 122 of the self-suction cover 12 is from 10A to 50A, so as to improve comfort while ensuring the flexibility of the horn cover 122. The surface roughness range of the inner wall of the horn cover 122 of the self-suction cover 12 is Ra0.016 um-Ra0.063 um, so that when the horn cover 122 is adsorbed onto the breast, there is a reasonable frictional force between the horn cover 122 and the breast, so that the self-suction cover 12 can stably attach to the breast.

Preferably, referring to FIG. 4, the distance L1 between the front side of the top of the horn cover 122 and the rear side of the assembly ring 121 of the self-suction cover 12 is greater than the distance L2 between the front side of the bottom of the horn cover 122 and the rear side of the assembly ring 121. That is, on the front side of the horn cover 122, the top of the horn cover 122 is protruded further forward than the bottom, so that the contact area between the horn cover 122 and the upper part of the breast is greater than the contact area between the horn cover 122 and the lower part of the breast. In this way, the shape of the horn cover 122 is more in line with the shape of the breast, which helps to increase the friction between the horn cover 122 and the breast, so that the self-suction breast pumping device 10 can be more firmly attached to the breast.

Furthermore, in the self-suction breast pumping device 10 of the present invention, the negative pressure forming unit 11 does not require any electronic components. Instead, the air exchange channel 1102 of the negative pressure forming unit 11 is connected to the main unit 20 via an air tube 30. This allows the self-suction breast pumping device 10 to have a lighter weight and smaller size. This not only allows the center of gravity of the self-suction breast pumping device 10 to be closer to the self-suction cover 12, ensuring reliable adhesion to the breast, but also reduces the burden on the breast due to the lighter weight. Simultaneously, because the negative pressure forming unit 11 has a smaller size, even if the user chooses to use a bra to assist in attaching the self-suction breast pumping device 10 to the breast, the negative pressure forming unit 11 will not excessively stretch the bra outwards. At the same time, the pressure exerted on the breast by the bra through the negative pressure forming unit 11 is reduced, improving the user's comfort when pumping and promoting breast health.

Referring again to FIG. 4, the self-suction cover 12 comprises a small cover 123 which is disposed at the connection position between the horn cover 122 and the assembly ring 121, and at least a portion of the small cover 123 is surrounded by the horn cover 122. In this way, when the user attaches the self-suction breast pumping device 10 to the breast, the inner wall of the small cover 123 fits against the areola in such a way that the small cover 123 covers the nipple, ensuring that the milk suction chamber 1103 of the negative pressure forming unit 11 forms a closed space and ensuring the sealing of the negative pressure space 100 formed between the breast and the horn cover 122, thereby allowing the self-suction breast pumping device 10 to be reliably attached to the breast.

It is worth noting that the specific manner in which the small cover 123 is positioned at the connection point between the horn cover 122 and the assembly ring 121 is not limited in the breast pump of the present invention. For example, in this specific example of the breast pump shown in FIGS. 1 to 7, the small cover 123 may be extended forward integrally from the connection point between the horn cover 122 and the assembly ring 121 to position the small cover 123 at the connection point. Optionally, the small cover 123 of the breast pump of the present invention is detachable.

In this specific example of the breast pump of the present invention shown in FIGS. 1 to 7, the assembly ring 121 of the self-suction cover 12 is detachably mounted to the negative pressure forming part 112, which allows the self-suction cover 12 to be easily cleaned, thereby improving the hygiene of the breast pump. At the same time, mothers can choose the appropriate material, shape, and/or size of the self-suction cover 12 according to their own needs to meet individual requirements.

Specifically, the outer diameter of the assembly ring 121 of the self-suction cover 12 is the same as the inner diameter of the negative pressure forming part 112. Therefore, after the assembly ring 121 of the self-suction cover 12 is installed on the negative pressure forming part 112, the self-suction cover 12 can be reliably installed on the negative pressure forming unit 11 based on the friction between the outer wall of the assembly ring 121 and the inner wall of the negative pressure forming unit 112.

Referring to FIGS. 4 to 7, after the assembly ring 121 of the self-suction cover 12 is installed on the negative pressure forming part 112, the rear side of the assembly ring 121 is protruded above the opening formed on the inner wall of the deformable bowl 1121 by the milk discharge channel 1105 of the negative pressure forming unit 11. In this way, the sucked milk can be blocked by the assembly ring 121 of the self-suction cover 12, preventing the milk from flowing to the position of the horn cover 122, thereby preventing the milk from leaking from the position where the horn cover 122 and the breast are in contact. That is, the milk is only allowed to be discharged to the milk bottle 14 through the milk discharge channel 1105 of the negative pressure forming unit 11.

Referring again to FIG. 4, the self-suction cover 12 further comprises a limiting portion 124. The limiting portion 124 is extended from the peripheral wall of the assembly ring 121 in a direction substantially perpendicular to the extending direction of the assembly ring 121, and there is a gap between the limiting portion 124 and the rear portion 1221 of the horn cover 122. In this way, when the self-suction cover 12 is assembled at the assembly opening 1104 of the negative pressure forming unit 11, the limiting portion 124 restricts the assembly depth of the assembly ring 121 by abutting against the frame 1122 of the negative pressure forming portion 112. Thus, on the one hand, the limiting portion 124 can ensure the formation of the gap between the rear portion 1221 of the cover 122 and the negative pressure forming unit 11, so that when the assembly ring 121 changes position with the negative pressure forming unit 11, the horn cover 122 can deform without detaching from the breast. On the other hand, the limiting portion 124 ensures that the assembly ring 121 extends through the mounting opening 1104 of the negative pressure forming unit 11 to the outside of the opening formed by the milk discharge channel 1105 of the negative pressure forming unit 11 in the inner wall of the deformable bowl 1121, preventing the assembly ring 121 from blocking the opening formed by the milk discharge channel 1105 of the negative pressure forming unit 11 in the inner wall of the deformable bowl 1121. Preferably, the limiting portion 124 of the self-suction cover 12 is annular.

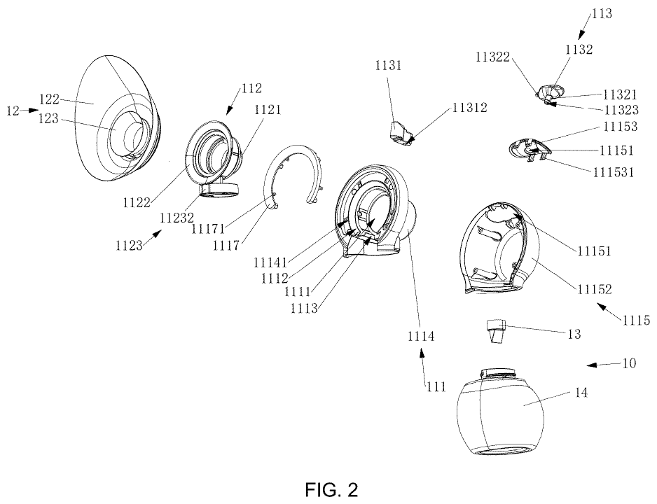

Referring to FIGS. 2, 3, and 4, the supporting housing 111 comprises a housing body 1114 and an outer cover 1115, and the supporting housing 111 has a housing receiving cavity 1116. The housing cavity 1111, the housing opening 1112, and the housing notch 1113 of the supporting housing 111 are formed in the housing body 1114. The outer cover 1115 is installed on the housing body 1114. The supporting housing 111 forms the housing receiving cavity 1116 between the housing body 1114 and the outer cover 1115. The outer cover 1115 has a positioning hole 11151 which is communicated with the housing receiving cavity 1116 of the supporting housing 111.

It is worth noting that the specific manner in which the housing body 1114 and the outer cover 1115 are mounted to each other is not limited in the breast pump of the present invention. For example, in this specific example of the breast pump of the present invention shown in FIGS. 1 to 7, screws can be used to mount the housing body 1114 and the outer cover 1115.

Preferably, the supporting housing 111 comprises a decorative cover 1117 which is installed on the housing body 1114 to cover the screws used to install the housing body 1114 and the outer cover 1115. More preferably, the decorative cover 1117 comprises a plurality of latching arms 11171, and the housing body 1114 has a plurality of latching holes 11141. The latching holes 11141 of the housing body 1114 are communicated with the housing receiving cavity 1116 of the supporting housing 111. Each latching arm 11171 of the decorative cover 1117 is extended into the housing receiving cavity 1116 of the supporting housing 1114 after passing through the latching holes 11141 of the housing body 1114. Within the housing receiving cavity 1116 of the supporting housing 1114, each latching arm 11171 of the decorative cover 1117 automatically latches onto the housing body 1114, thereby installing the decorative cover 1117 onto the housing body 1114.

Referring to FIGS. 2 to 4, the negative pressure forming unit 11 comprises an airway unit 113 which comprises an air guide base 1131 and a reversing base 1132.

The air guide base 1131 has a laterally extending first air passage 11311, a bottom insertion hole 11312, a top insertion hole 11313, and a top boss 11314. The bottom insertion hole 11312 and the top insertion hole 11313 are respectively communicated to the first air passage 11311, and the opening of the top insertion hole 11313 is formed on the end face of the top boss 11314. The housing body 1114 includes a housing insertion arm 11142 which is extended into the housing receiving cavity 1116 of the supporting housing 111. The opening of the air exchange channel 1102 of the negative pressure forming unit 11 is formed on the end face of the housing insertion arm 11142 of the housing body 1114. The air guide base 1131 is disposed on the top of the housing body 1114. The housing insertion arm 11142 of the housing body 1114 is inserted into the bottom insertion hole 11312 of the air guide base 1131. The top boss 11314 of the air guide base 1131 is extended into the positioning hole 11151 of the outer cover 1115. In this way, the air guide base 1131 can be reliably received in the housing receiving cavity 1116 of the supporting housing 111, and the first air passage 11311 of the air guide base 1131 is communicated to the air exchange channel 1102 of the negative pressure forming unit 11.

The reversing base 1132 has a bottom insert arm 11321, a side insert arm 11322 and a second air passage 11323. One opening of the second air passage 11323 is formed on the end face of the bottom insert arm 11321 and the other opening is formed on the end face of the side insert arm 11322. The bottom insert arm 11321 of the reversing base 1132 is inserted into the top insertion hole 11313 of the air guide base 1131, so that the negative pressure chamber 1101 of the negative pressure forming unit 11 and the air exchange channel 1102, the first air passage 11311 of the air guide base 1131 and the second air passage 11323 of the reversing base 1132 are sequentially connected. One end of the air pipe 30 is installed in the side insert arm 11322 of the reversing base 1132. In this way, the main unit 20 can change the air pressure state of the negative pressure chamber 1101 of the negative pressure forming unit 11 through the air pipe 30.

In this specific example of the breast pump of the present invention shown in FIGS. 1 to 7, by providing the air guide base 1131 and the reversing base 1132 on the top of the housing body 1114 of the supporting housing 111, the portion of the negative pressure forming unit 11 used to connect the air tube 30 (i.e., the side insert arm 11322 of the reversing base 1132) can be positioned reasonably, avoiding this portion occupying a large space. In other words, the arrangement of the air guide base 1131 and the reversing base 1132 of the airway unit 113 enables the miniaturization of the self-suction breast pumping device 10. Thus, even if the user chooses to use a bra to assist in adhering the self-suction breast pumping device 10 to the breast, the negative pressure forming unit 11 of the self-suction breast pumping device 10 will not excessively stretch the bra outward. At the same time, the pressure applied to the breast by the bra through the negative pressure forming unit 11 will be reduced, thereby improving the user's comfort when pumping milk with the breast pump and benefiting breast health.

In this specific example of the breast pump of the present invention shown in FIGS. 1 to 7, the bottom insertion hole 11312 of the reversing base 1132 is pluggably inserted into the top insertion hole 11313 of the air guide base 1131. This allows the user to change the orientation of the side insertion arm 11322 of the reversing base 1132 as needed. For example, the user can allow the bottom insert arm 11321 of the reversing base 1132 to be positioned at the side of the reversing base 1132 as needed. The side insert arm 11322 is inserted into the top insertion hole 11313 of the air guide base 1131 with the left-facing side, or the bottom insert arm 11321 of the reversing base 1132 is inserted into the top insertion hole 11313 of the air guide base 1131 with the side insert arm 11322 of the reversing base 1132 facing the right. In this way, the user does not need to distinguish the orientation of the two self-suction breast pumps 10 during the assembly of the breast pump.

It is understood that, since the housing insertion arm 11142 of the housing body 1114 of the supporting housing 111 is inserted into the bottom insertion hole 11312 of the air guide base 1131 and the top boss 11314 of the air guide base 1131 is extended into the positioning hole 11151 of the outer cover 1115, the air guide base 1131 can be stably received in the housing receiving cavity 1116 of the supporting housing 111. Therefore, whether the bottom insert arm 11321 of the reversing base 1132 is inserted into the top insertion hole 11313 of the air guide base 1131 or the bottom insert arm 11321 of the reversing base 1132 is pulled out from the top insertion hole 11313 of the air guide base 1131, the air guide base 1131 will not shift, thus ensuring the reliability of the breast pump.

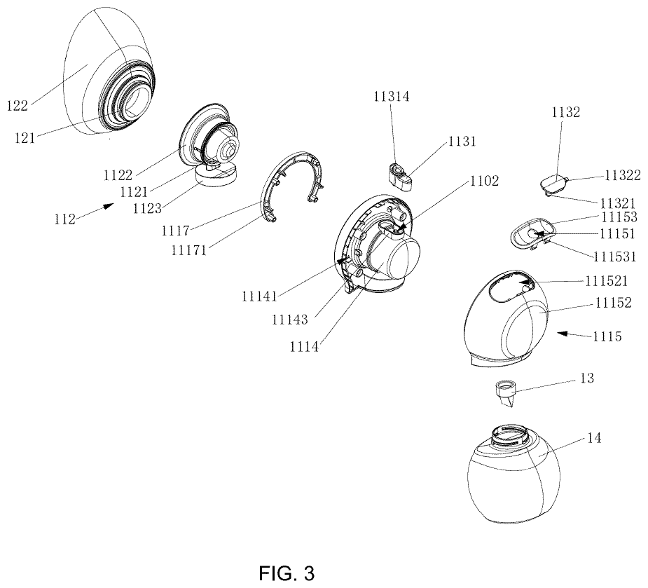

Referring to FIGS. 3 and 4, the top of the housing body 1114 has a positioning wall 11143 which surrounds the bottom of the air guide base 1131, and the positioning wall 11143 is in close contact with the outer wall of the air guide base 1131. Thus, on the one hand, before assembling the outer cover 1115 onto the housing body 1114, the positioning wall 11143 of the housing body 1114 pre-positions the air guide base 1131; when assembling the outer cover 1115 onto the housing body 1114, the positioning wall 11143 of the housing body 1114 can prevent the displacement of the air guide base 1131, so as to ensure that the top boss 11314 of the air guide base 1131 is inserted into the positioning hole 11151 of the outer cover 1115, thereby improving assembly efficiency. On the other hand, when the bottom insert arm 11321 of the reversing base 1132 is inserted into the top insertion hole 11313 of the air guide base 1131 or the bottom insert arm 11321 of the reversing base 1132 is pulled out from the top insertion hole 11313 of the air guide base 1131, the positioning wall 11143 of the housing body 1114 can prevent the air guide base 1131 from shifting.

Referring again to FIGS. 2 to 4, the outer cover 1115 further comprises an outer cover body 11152 and an anti-detachment cover 11153. The positioning hole 11151 is formed in the anti-detachment cover 11153. The outer cover body 11152 has an outer cover channel 111521. The outer cover body 11152 is fixedly mounted on the housing body 1114. The position of the top boss 11314 of the air guide base 1131 corresponds to the outer cover channel 111 of the outer cover body 11152. The anti-detachment cover 11153 is fixedly installed in the outer casing channel 111521 of the outer cover body 11152, and the top boss 11314 of the air guide base 1131 is inserted into the positioning hole 11151 formed in the anti-detachment cover 11153 of the outer cover 1115. In this way, the outer casing 1114, the air guide base 1131 and the outer cover 1115 can be easily assembled, and the misalignment of the air guide base 1131 is avoided.

It is worth mentioning that the specific manner in which the anti-detachment cover 11153 is fixedly installed in the outer cover channel 111521 of the outer cover body 11152 is not limited to the breast pump of the present invention. For example, in this specific example of the breast pump of the present invention shown in FIGS. 1 to 7, referring to FIGS. 2 to 4, the anti-detachment cover 11153 has a plurality of hook arms 111531 on each opposite side, the shape of the anti-detachment cover 11153 matches the shape of the outer cover channel 111521 of the outer cover body 11152, and the size of the anti-detachment cover 11153 is slightly larger than the size of the outer cover channel 111521 of the outer cover body 11152. The hook arms 111531 are extended into the housing receiving cavity 1116 of the supporting housing 111 through the outer cover channel 111521 of the outer cover body 11152, and the hook arms 111531 of the anti-detachment cover 11153 hook the edge of the outer cover body 11152 that forms the outer cover channel 111521, so as to reliably install the anti-detachment cover 11153 onto the outer cover body 11152 and close the outer cover channel 111521 of the outer cover body 11152.

According to another aspect of the present invention, the present invention further provides a method for operating the self-suction breast pumping device 10. Referring to FIGS. 5 to 7, the method comprises the following steps:
(a) Flip the horn cover 122 of the self-suction cover 12 toward the negative pressure forming unit 11;
(b) Allow the position of the horn cover 122 of the self-suction cover 12 for connecting the assembly ring 121 to abut against the areola; and
(c) Restore the horn cover 122, so that the horn cover 122 covers a portion of the breast to form the negative pressure space 100 between the horn cover 122 and the breast, so as to allow the self-suction breast pumping device 10 to attach to the breast.

After the self-suction breast pumping device 10 is attached to the breast, the breast closes the front opening of the horn cover 122, and the areola closes the rear opening of the horn cover 122. Thus, the self-suction breast pumping device 10 not only allows the negative pressure space 100 to be formed between the horn cover 122 and the breast, but also allows the milk suction chamber 1103 of the negative pressure forming unit 11 to form a closed space. Through this operating method, the self-suction breast pumping device 10 can be reliably attached to the breast without the need for underwear, enabling breastfeeding without underwear.

Preferably, in the step (b), the operating method allows the inner wall of the small cover 123 of the self-suction cover 12 to fit against the areola in such a way that the small cover 123 covers the nipple. In this way, it is beneficial to ensure that the milk suction chamber 1103 of the negative pressure forming unit 11 forms a closed space, thereby increasing the sealing of the negative pressure space 100, so that the self-suction milk suction device 10 can be reliably adsorbed onto the breast.

Preferably, in the step (b), the contact area between the horn cover 122 of the self-suction cover 12 and the upper part of the breast is greater than the contact area between the horn cover 122 and the lower part of the breast. This makes the shape of the horn cover 122 more closely match the shape of the breast, which helps increase the friction between the horn cover 122 and the breast, allowing the self-suction breast pumping device 10 to adhere more firmly to the breast. More preferably, the self-suction cover 12 is made of silicone. Because the distance between the front side of the top of the horn cover 122 and the rear side of the assembly ring 121 is greater than the distance between the front side of the bottom of the horn cover 122 and the rear side of the assembly ring 121, the horn cover 122 can provide greater wrapping force on the upper part of the breast due to the extensibility of silicone, so as to allow the self-suction breast pumping device 10 to adhere more firmly to the breast.

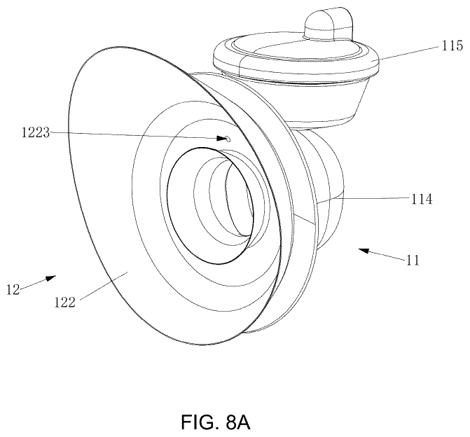

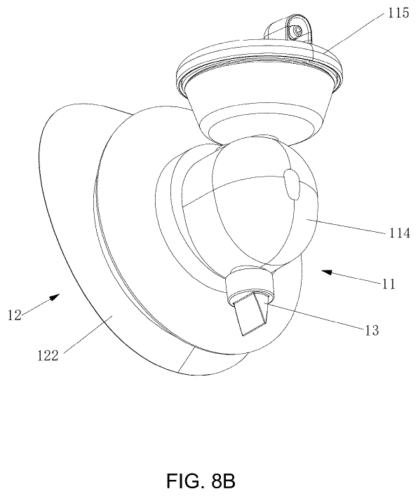

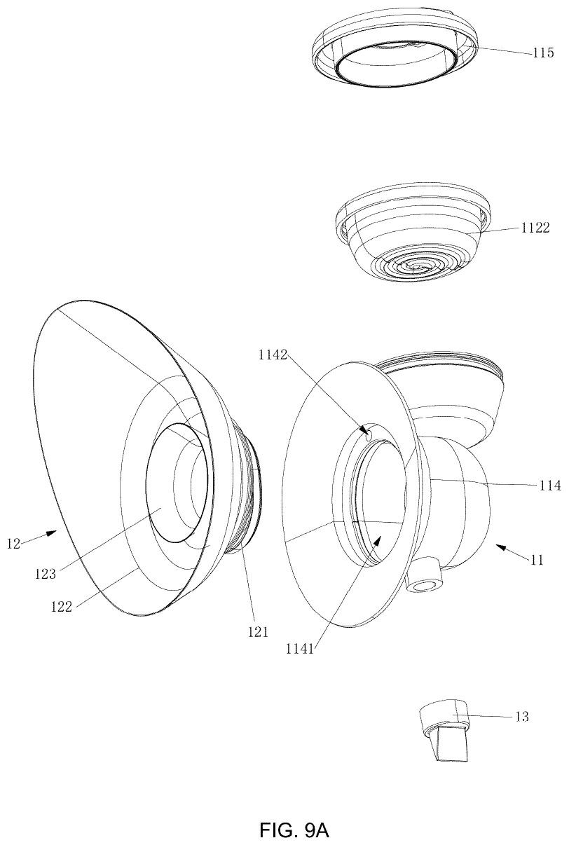

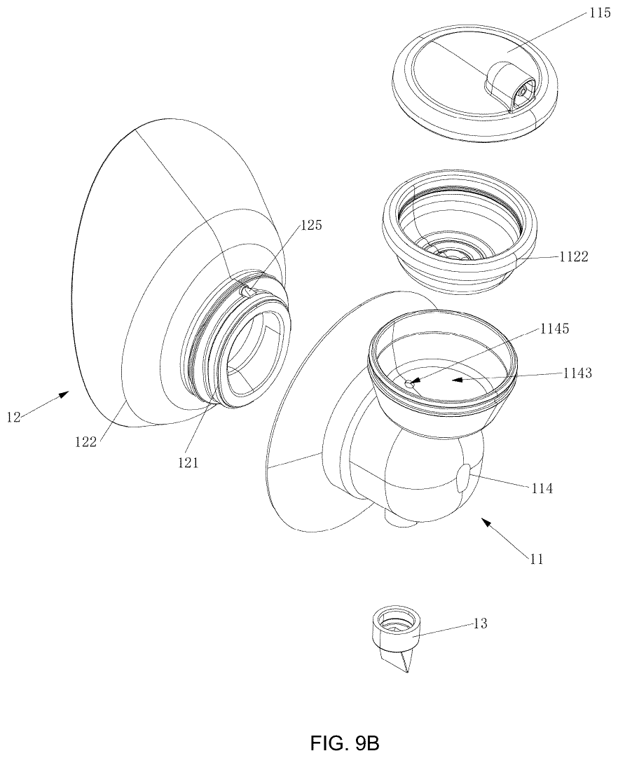

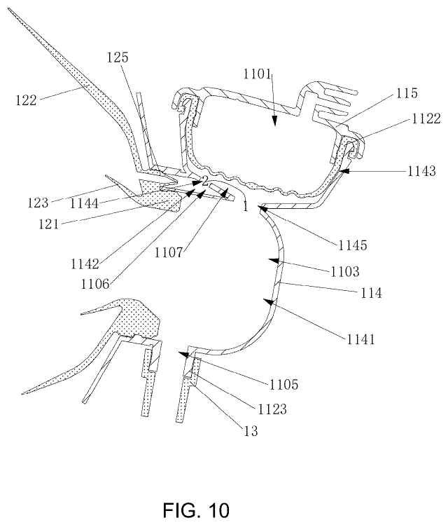

FIGS. 8A to 10 show a modified example of the self-suction breast pumping device 10. Unlike the self-suction breast pumping device 10 of the breast pump shown in FIGS. 1 to 7, in this specific example of the self-suction breast pumping device 10 of the present invention shown in FIGS. 8A to 10, the horn cover 122 of the self-suction cover 12 has an exhaust passage 1223. The self-suction cover 12 further comprises an exhaust one-way valve 125 which is disposed on the horn cover 122 for selectively opening and closing the exhaust passage 1223 of the horn cover 122. When the self-suction breast pumping device 10 is attached to the breast to form the negative pressure space 100 between the horn cover 122 and the breast, the exhaust one-way valve 125 allows air within the negative pressure space 100 to be discharged through the exhaust channel 1223 of the horn cover 122. This ensures that the air pressure within the negative pressure space 100 is lower than the external ambient air pressure, thereby guaranteeing that the self-suction breast pumping device 10 reliably attaches to the breast. Preferably, the exhaust one-way valve 125 and the horn cover 122 are integrally formed.

Furthermore, the negative pressure forming unit 11 has a valve chamber 1106 and a connecting channel 1107. The valve chamber 1106 is located above the milk suction chamber 1103, and the connecting channel 1107 connects the valve chamber 1106 with the milk suction chamber 1103. The end of the exhaust one-way valve 125 facing away from the horn cover 122 is extended into the valve chamber 1106 of the negative pressure forming unit 11. It can be understood that when the self-suction milk suction device 10 is attached to the breast, the negative pressure space 100 is formed between the horn cover 122 and the breast. Simultaneously, the areola closes the rear opening of the horn cover 122, thus forming a closed space for the milk suction chamber 1103 of the negative pressure forming unit 11. Therefore, the negative pressure space 100 and the milk suction chamber 1103 of the negative pressure forming unit 11 are independent of each other. When the main unit 20 draws in air for causing a decrease in the air pressure of the milk suction chamber 1103 of the negative pressure forming unit 11, the exhaust one-way valve 125 opens the exhaust passage 1223 of the horn cover 122 to allow air in the negative pressure space 100 to enter the milk suction chamber 1103 of the negative pressure forming unit 11 through the exhaust passage 1223 of the horn cover 122. This prevents the horn cover 122 from detaching from the breast during milk suction, ensuring that the self-suction milk suction device 10 is reliably attached to the breast. Conversely, when the main unit 20 exhausts air, causing an increase in the air pressure of the milk suction chamber 1103 of the negative pressure forming unit 11, the exhaust one-way valve 125 closes the exhaust passage 1223 of the horn cover 122 to prevent air in the milk suction chamber 1103 of the negative pressure forming unit 11 from entering the negative pressure space 100, thus ensuring that the self-suction milk suction device 10 is reliably attached to the breast.

In the breast pump of the present invention, since the valve chamber 1106 of the negative pressure forming unit 11 is located above the milk suction chamber 1103, and the valve chamber 1106 and the milk suction chamber 1103 are connected through the connecting channel 1107, and the connecting channel 1107 has a small size, the milk being suctioned is prevented from entering the valve chamber 1106 from the milk suction chamber 1103 through the connecting channel 1107 of the negative pressure forming unit 11 during the milk suction process.

Preferably, the air flow rate at the connection point between the connecting channel 1107 and the valve chamber 1106 of the negative pressure forming unit 11 is less than the air flow rate at the connection point between the connecting channel 1107 and the milk suction chamber 1103. In this way, it can be ensured that the air pressure in the milk suction chamber 1103 of the negative pressure forming unit 11 drops low enough to provide strong suction for milk suction, while avoiding a significant drop in air pressure in the valve chamber 1106 of the negative pressure forming unit 11. As a result, the horn cover 122 will not deform excessively, thus ensuring comfort during milk suction.

Preferably, the exhaust one-way valve 125 is disposed on the rear side of the top of the horn cover 122. In this way, the exhaust one-way valve 125 and the valve chamber 1106 of the negative pressure forming unit 11 cooperate with each other to guide the installation method of the self-suction cover 12. That is, after the self-suction cover 12 is installed on the negative pressure forming unit 11, the forward-protruding part of the horn cover 122 can automatically be located at the top, ensuring that the top of the horn cover 122 protrudes forward more than the bottom.

Unlike the specific structure of the negative pressure forming unit 11 of the self-suction breast pumping device 10 shown in FIGS. 1 to 7, in this specific example of the negative pressure forming unit 11 shown in FIGS. 8A to 10, the negative pressure forming unit 11 comprises a breast pump shell 114, a deformable bowl 1121, and a top cover 115. The breast pump shell 114 has a first recess 1141 with a front opening, a second recess 1142 with a front opening, a third recess 1143 with a top opening, a first through hole 1144, and a second through hole 1145. The second recess 1142 is located above the first recess 1141. The first through hole 1144 connects the first recess 1141 with the third recess 1143, and the second through hole 1145 connects the second recess 1142 with the third recess 1143. The deformable bowl 1121 is housed in the third recess of the breast pump shell 114, and the edge of the deformable bowl 1121 rests against the edge of the third recess 1143 of the breast pump shell 114. The communicating channel 1107 of the negative pressure forming unit 11 is formed between the breast pump shell 114 and the deformable bowl 1121. The first recess 1141 and the second recess 1142 of the breast pump shell 114 respectively form the milk suction chamber 1103 and the valve chamber 1106 of the negative pressure forming unit 11. The top cover 115 is installed on the breast pump shell 114, and the edge of the top cover 115 and the edge of the third recess 1143 of the breast pump shell 114 clamp the edge of the deformable bowl 1121 to form the negative pressure chamber 1101 of the negative pressure forming unit 11 between the top cover 115 and the deformable bowl 1121, wherein the air tube 30 can be connected to the top cover 115.

The size of the first through hole 1144 of the breast pump shell 114 is larger than the size of the second through hole 1145, so that the air flow rate at the connection position of the connecting channel 1107 and the valve chamber 1106 of the negative pressure forming unit 11 is less than the air flow rate at the connection position of the connecting channel 1107 and the milk suction chamber 1103.

Referring again to FIGS. 8A to 10, the negative pressure forming unit 11 further comprises a mounting arm 1123 which is extended integrally downward from the breast pump shell 114. The milk discharge channel 1105 of the negative pressure forming unit 11 is extended from the inner wall of the breast pump shell 114 to the end of the mounting arm 1123. The milk discharge one-way valve 13 is mounted on the mounting arm 1123 to selectively open and close the milk discharge channel 1105 of the negative pressure forming unit 11.

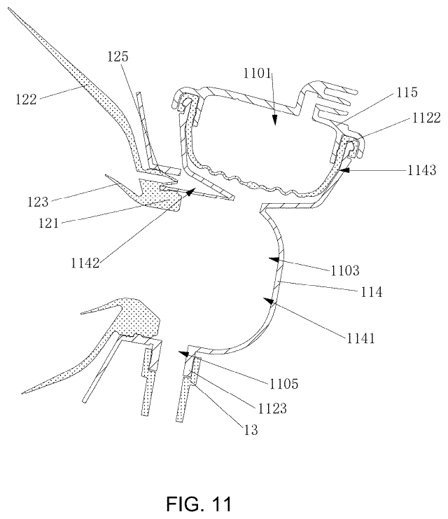

FIG. 11 shows another variation of the self-suction breast pumping device 10. Unlike the self-suction breast pumping device 10 shown in FIGS. 8A to 10, in this specific example of the self-suction breast pumping device 10 shown in FIG. 11, the end of the exhaust one-way valve 125 of the self-suction cover 12, which is away from the self-suction cover 122, is located outside the negative pressure forming unit 11. That is, when the self-suction breast pumping device 10 is attached to the breast and forms the negative pressure space 100 between the horn cover 122 and the breast, the exhaust one-way valve 125 allows the air in the negative pressure space 100 to be discharged to the external environment through the exhaust channel 1223 of the horn cover 122. This ensures that the air pressure in the negative pressure space 100 is lower than the air pressure in the external environment, thereby ensuring that the self-suction breast pumping device 10 is reliably adsorbed onto the breast.

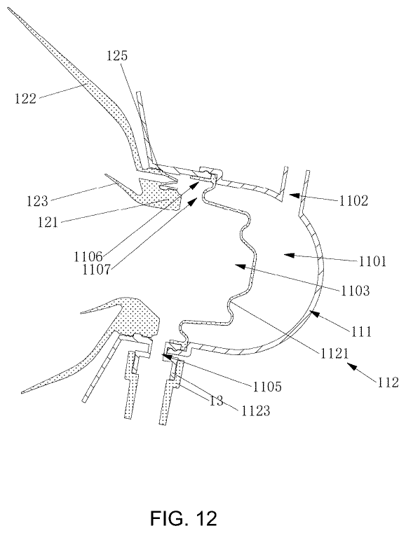

FIG. 12 shows a modified example of the self-suction breast pumping device 10. Unlike the self-suction breast pumps 10 shown in FIGS. 1 to 7, in this specific example of the self-suction breast pumping device 10 of the present invention shown in FIG. 12, the horn cover 122 of the self-suction cover 12 has the exhaust channel 1223. The self-suction cover 12 comprises the exhaust one-way valve 125 which is disposed on the horn cover 122 for selectively opening and closing the exhaust channel 1223 of the horn cover 122. The negative pressure forming unit 11 has the valve chamber 1106 and the connecting channel 1107, the connecting channel 1107 connecting the milk suction chamber 1103 with the valve chamber 1106, the valve chamber 1106 and the connecting channel 1107 are formed in the negative pressure forming part 112, when the self-suction cover 12 is installed in the negative pressure forming part 112, the exhaust one-way valve 125 of the self-suction cover 12 is extended into the valve chamber 1106 of the negative pressure forming unit 11. When the main unit 20 draws in air, causing a decrease in the air pressure of the milk suction chamber 1103 of the negative pressure forming unit 11, the exhaust one-way valve 125 opens the exhaust channel 1223 of the horn cover 122 to allow air in the negative pressure space 100 to enter the milk suction chamber 1103 of the negative pressure forming unit 11 through the exhaust channel 1223 of the horn cover 122. This prevents the horn cover 122 from detaching from the breast during milk suction, ensuring that the self-suction breast pumping device 10 is reliably attached to the breast. Conversely, when the main unit 20 exhausts air, causing an increase in the air pressure of the milk suction chamber 1103 of the negative pressure forming unit 11, the exhaust one-way valve 125 closes the exhaust passage 1223 of the horn cover 122 to prevent air in the milk suction chamber 1103 of the negative pressure forming unit 11 from entering the negative pressure space 100, thus ensuring that the self-suction milk suction device 10 is reliably attached to the breast.

Those skilled in the art should understand that the embodiments of the present invention described above and shown in the accompanying drawings are merely examples and do not limit the present invention. The objectives of the present invention have been fully and effectively achieved. The functions and structural principles of the present invention have been demonstrated and explained in the embodiments, and any variations or modifications may be made to the implementation of the present invention without departing from the stated principles.

What is claimed is:

1. A self-suction breast pumping device, comprising;
a milk discharge one-way valve;
a self-suction cover, wherein the self-suction cover comprises an assembly ring and a horn cover which is deformable and extended outward from the assembly ring; and
a negative pressure forming unit, wherein the negative pressure forming unit has a negative pressure chamber, an air exchange channel communicated with the negative pressure chamber, a milk suction chamber independent of the negative pressure chamber, and an assembly opening and a milk discharge channel communicated with the milk suction chamber, wherein a volume size of the negative pressure chamber is negatively correlated with a volume size of the milk suction chamber, wherein the milk discharge one-way valve is disposed in the negative pressure forming unit for selectively opening and closing the milk discharge channel of the negative pressure forming unit, wherein the assembly ring of the self-suction cover is assembled to the negative pressure forming unit at the assembly opening.

2. The self-suction breast pumping device according to claim 1, wherein the horn cover comprises a rear portion and a front portion, the rear portion of the horn cover connects the front portion of the horn cover with the assembly ring, wherein an angle α is defined between an extending direction of the rear portion of the horn cover and a central axis of the self-suction cover, and an angle β is defined between an extending direction of the front portion of the horn cover and the central axis of the self-suction cover, wherein the angles α and β satisfy: α>β.

3. The self-suction breast pumping device according to claim 2, wherein the included angle β satisfies: 15°≤β≤40°.

4. The self-suction breast pumping device according to claim 1, wherein the self-suction cover further comprises a small cover which is extended from a connection position between the horn cover and the assembly ring, the small cover is provided at an inner side of the horn cover to cover a nipple.

5. The self-suction breast pumping device according to claim 1, wherein a distance between a front side of a top of the horn cover and a rear side of the assembly ring is greater than a distance between a front side of a bottom of the horn cover and the rear side of the assembly ring.

6. The self-suction breast pumping device according to claim 1, wherein the negative pressure forming unit comprises a supporting housing and a negative pressure forming part, the supporting housing has a housing cavity, a housing opening communicated to the housing cavity and a housing notch communicated to the housing cavity, the negative pressure forming part comprises a deformable bowl, an annular frame disposed at an opening of the deformable bowl, and a mounting arm extended downward from the annular frame, wherein the annular frame is mounted to the supporting housing at the housing opening, the deformable bowl is extended into the housing cavity of the supporting housing, the mounting arm is located at the housing notch of the supporting housing, wherein the negative pressure chamber of the negative pressure forming unit is formed between the supporting housing and the deformable bowl, wherein the air exchange channel of the negative pressure forming unit is formed in the supporting housing., the milk suction chamber and the assembly opening of the negative pressure forming unit are formed in the annular frame, the milk discharge channel of the negative pressure forming unit is extended from an inner wall of the deformable bowl through the annular frame to an end of the mounting arm or from the inner wall of the annular frame to the end of the mounting arm, the milk discharge one-way valve is mounted on the mounting arm.

7. The self-suction breast pumping device according to claim 6, wherein the self-suction cover further comprises a limiting portion extended from a peripheral wall of the assembly ring in a direction substantially perpendicular to an extending direction of the assembly ring, and a gap is formed between the limiting portion and the horn cover, wherein the limiting portion is capable of abutting against the annular frame of the negative pressure forming part.

8. The self-suction breast pumping device according to claim 6, wherein the mounting arm of the negative pressure forming part comprises an inner arm body and an outer arm body surrounding the inner arm body, wherein the milk discharge one-way valve is mounted on the inner arm body, wherein a milk bottle is mounted on the outer arm body.

9. The self-suction breast pumping device according to claim 6, wherein when an air pressure in the negative pressure chamber of the negative pressure forming unit decreases, the deformable bowl of the negative pressure forming part deforms toward a bottom of the housing cavity of the supporting housing to reduce an air pressure in the milk suction chamber of the negative pressure forming unit, wherein when the air pressure in the negative pressure chamber of the negative pressure forming unit increases, the deformable bowl of the negative pressure forming part deforms toward the bottom of the housing cavity of the supporting housing to increase the air pressure in the milk suction chamber of the negative pressure forming unit.

10. The self-suction breast pumping device according to claim 6, wherein the assembly ring of the self-suction cover is detachably mounted to the negative pressure forming part.

11. The self-suction breast pumping device according to claim 6, wherein an outer diameter of the assembly ring of the self-suction cover is the same as an inner diameter of the negative pressure forming part.

12. The self-suction breast pumping device according to claim 6, wherein after the assembly ring of the self-suction cover is installed on the negative pressure forming part, a rear side of the assembly ring is protruded above an opening formed on an inner wall of the deformable bowl by the milk discharge channel of the negative pressure forming unit, so as to allow the sucked milk to be blocked by the assembly ring of the self-suction cover and preventing the milk from flowing to the horn cover.

13. The self-suction breast pumping device according to claim 6, wherein the supporting housing comprises a housing body and an outer cover, and the supporting housing has a housing receiving cavity, wherein the housing cavity, the housing opening, and the housing notch of the supporting housing are formed in the housing body, the outer cover is installed on the housing body, the supporting housing forms the housing receiving cavity between the housing body and the outer cover, wherein the outer cover has a positioning hole that is communicated with the housing receiving cavity of the supporting housing.

14. The self-suction breast pumping device according to claim 13, wherein the negative pressure forming unit comprises an airway unit which comprises an air guide base and a reversing base, the air guide base has a first air passage which is laterally extending, a bottom insertion hole, a top insertion hole, and a top boss, the bottom insertion hole and the top insertion hole are respectively communicated with the first air passage, and an opening of the top insertion hole is formed on an end face of the top boss, the housing body comprises a housing insertion arm extended into the housing receiving cavity of the supporting housing, an opening of the air exchange channel of the negative pressure forming unit is formed on an end face of the housing insert arm of the housing body, wherein the air guide base is disposed on a top of the housing body, wherein the housing insert arm of the housing body is inserted into the bottom insertion hole of the air guide base, the top boss of the air guide base is extended into the positioning hole of the outer cover, the air guide base is received in the housing receiving cavity of the supporting housing, the first air passage of the air guide base is communicated to the air exchange channel of the negative pressure forming unit, the reversing base comprises a bottom insert arm, a side insert arm, and a second air passage, a first opening of the second air passage is formed on an end face of the bottom insert arm, and a second opening of the second air passage is formed on the end face of the side insert arm, wherein the bottom insert arm of the reversing base is inserted into the top insertion hole of the air guide base to allow the negative pressure chamber of the negative pressure forming unit and the air exchange channel, the first air passage of the air guide base, and the second air passage of the reversing base to be sequentially connected.

15. The self-suction breast pumping device according to claim 1, wherein when a space of the negative pressure chamber of the negative pressure forming unit increases, a space of the milk suction chamber decreases, wherein when the space of the negative pressure chamber of the negative pressure forming unit decreases, the space of the milk suction chamber increases, wherein an air pressure state of the negative pressure chamber of the negative pressure forming unit is positively correlated with an air pressure state of the milk suction chamber, wherein when an air pressure of the negative pressure chamber of the negative pressure forming unit increases, an air pressure of the milk suction chamber increases, and when the air pressure of the negative pressure chamber of the negative pressure forming unit decreases, the air pressure of the milk suction chamber decreases.

16. The self-suction breast pumping device according to claim 1, wherein the horn cover of the self-suction cover is configured to be flipped toward the negative pressure forming unit to allow a position of the horn cover for connecting the assembly ring to contact an areola, wherein the horn cover is configured to be restored to cover a part of a breast, thereby forming a negative pressure space between the horn cover and the breast, so as to allow the self-suction breast pumping device to attach to the breast and achieve naked breast pumping in a manner that the self-suction breast pumping device is attached to the breast while the breast and the areola are respectively closing two ends of the horn cover.

17. The self-suction breast pumping device according to claim 16, wherein the self-suction cover is made of silicone.

18. The self-suction breast pumping device according to claim 15, wherein when air in the negative pressure chamber of the negative pressure forming unit is discharged through the air exchange channel to reduce the air pressure and space of the negative pressure chamber, the space of the milk suction chamber of the negative pressure forming unit increases and the air pressure thereof decreases, so that milk is sucked out of the breast and temporarily stored in the milk suction chamber of the negative pressure forming unit, wherein a position of the horn cover for connecting the assembly ring prevents the areola from being pulled toward the milk suction chamber of the negative pressure forming unit, so as to allow the position of the horn cover for connecting the assembly ring to squeeze the areola to squeeze out the milk, wherein when air is filled into the negative pressure chamber of the negative pressure forming unit to increase the air pressure and space of the negative pressure chamber, the space of the milk suction chamber of the negative pressure forming unit decreases and the air pressure thereof increases, wherein the milk discharge one-way valve automatically opens to discharge the milk temporarily stored in the milk suction chamber of the negative pressure forming unit.

19. The self-suction breast pumping device according to claim 17, wherein a hardness range of the horn cover of the self-suction cover is from 10A to 50A.

20. The self-suction breast pumping device according to claim 19, wherein a surface roughness range of an inner wall of the horn cover of the self-suction cover is Ra0.016 um-Ra0.063 um.
